(19) United States
(12) Patent Application Publication
(10) Pub. No.: US 2008/0138799 A1
(43) Pub. Date: Jun. 12, 2008

(54) Method and a system for extracting a genotype-phenotype relationship

(75) Inventors: Jie Cheng, Princeton, NJ (US); Mathaeus Dejori, Munich (DE); Martin Stetter, Munich (DE); Bernd Wachmann, Lawrenceville, NJ (US)

Correspondence Address: STAAS & HALSEY LLP, SUITE 700, 1201 NEW YORK AVENUE, N.W., WASHINGTON, DC, 20005, US

(73) Assignee: SIEMENS AKTIENGESELLSCHAFT, Munich (DE)

(21) Appl. No.: 11/247,218

(22) Filed: Oct. 12, 2005

(51) Int. Cl. C12Q 1/68; G06F 19/00

(52) U.S. Cl. 435/6; 702/20

(57) ABSTRACT

At least one genotype-phenotype relationship is extracted based on genotype data of a group of genes for different organisms of a group of organisms. A first database stores genotype data of each organism of the group of organisms. For each organism a genotype vector is stored having a vector component for each gene of the group of genes. A second database stores phenotype data of each organism of the group of organisms. For each organism a phenotype vector is stored having a vector component for each phenotype feature of a group of phenotype features of the organism. A calculation unit uses a machine learning process to classify organisms with different phenotypes depending on the genotype vectors stored in the first database and the phenotype vectors stored in the second database to extract the genotype-phenotype relationship.

### BACKGROUND OF THE INVENTION

The invention relates in general to a method for extracting at least one genotype-phenotype relationship on the basis of genotype data of a group of genes for different organisms.

Individual alterations related to organisms in the nucleotide sequences of their respective DNA (Deoxyribonucleic Acid) causes changes in the metabolic pathways of such an organism which can be quantified by modifications of concentrations and structures of RNAs and proteins. These changes in the metabolic pathways of said organism can subsequently lead to diseases or individually different responses of said organism to drug treatments.

The DNA sequence is a succession of letters representing the primary structure of a real DNA molecule or a strand. The possible letters A, C, G, T represent four nucleotide sub-units of a DNA strand, i. e. adenine, cytosine, guanine and thymine. The strand of DNA contains genes, areas that regulate genes and areas that have no function. The DNA is organized in two complementary strands with weak chemical bonds between them. Each base forms hydrogen bonds readily to another base, i. e. A to T and C to G. Since there are just four possible combinations, naming only one base on one side of the strand is enough to describe the DNA sequence. The order of the bases along the length of the DNA strand is a description of the genes.

A gene sequence of nucleotides along the DNA strand defines a messenger RNA sequence which in turn defines a protein pattern liable to be manufactured or “expressed” using this information encoded in the sequence. The relationship between the nucleotide sequence and the amino-acid sequence of the protein is determined by cellular rules of translation, known collectively as the genetic code. A genetic code is made of three letter “words” which are also termed codons formed from the sequence of three nucleotides. These codons are translated with a messenger RNA and a transfer RNA, with a codon corresponding to a particular amino-acid. Since there are 64 possible codons most amino-acids have more than one possible codon.

A genetic polyphormism is a occurrence of a gene variation, i. e. an allele variation. The replication of a DNA is performed by splitting the double-strand down the middle and recreating the other half of each new single strand by drowning each half in an environment containing the four basic bases. Since each of the bases can only combine with another base the base on the old strand dictates which base will be on the new strand. Mutations are chemical imperfections in this process when a base is accidentally skipped, inserted or incorrectly copied. A type of sequence variation is the so-called Single-Nucleotide Polymorphism SNP wherein one nucleotide in the DNA sequence is exchanged. When the SNP is in an area of a coding sequence, i. e. within an area of a gene, this can result to an exchange of an amino-acid in the resulting protein.

A genotype-phenotype distinction of organism refers to the fact that, while genotype and phenotype of an organism are related, they do not necessarily coincide. The genotype of an organism represents its exact genetic make-up, i. e. a particular set of genes it possesses. Two organisms whose genes differ at even one locus, i. e. at one position in said genome, have different genotypes. The term “genotype” refers to the full hereditary information of the organism. The phenotype of an organism, on the other hand represents its actual physical properties, such as height, weight, hair colour etc. Although the organism's genotype is the largest influencing factor in the development of its phenotype it is not the only one because the environment also influences the development of the phenotype of an organism.

Many diseases of an organism including diseases such as cancer are known to be genetically caused or influenced. As an effect of a local change in the function of one or a small collection of genes, the whole genetic program and the operational mode of a cell turns into a pathological mode. This transformation is also paralleled by a change in the global gene expression profile. However, in many cases it is unknown which genetic changes cause a disease and how the genetic change results via the genetic network of the organism in the pathological change.

Since alteration in the nucleotide sequences of the DNA causes changes in the genetic, protein and metabolic pathways of said organism leading to modifications of concentrations and structures of RNAs and proteins, it is the object of the present invention to identify patterns of these alterations which are statistically significant and causally related to physiological changes under predefined conditions.

### SUMMARY OF THE INVENTION

The present invention provides a method for extracting at least one genotype-phenotype relationship on the basis of genotype data of a group of genes for different organisms of a group of organisms. Genotype data of each organism of said group of organisms is input as a genotype polymorphism vector having at least one vector component for each gene of said group of genes. Phenotype data of each organism of said group of organisms is input as a phenotype vector having a vector component for each phenotype feature of a group of phenotype features of said organism. By a machine learning process, organisms with different phenotypes are classified depending on said input genotype vectors and said phenotype vectors to extract the genotype-phenotype relationship. The method according to the present invention allows the identification of genetic patterns via a differential diagnostic approach on the basis of a representative population of organisms. Based on statistical significance, the genetic patterns are then identified as relevant, depending on the level of abnormality they cause based on the underlying genotype data.

The method according to the present invention allows the integration of public databases storing genotype or phenotype data.

In one embodiment of the method according to the present invention, the machine learning process is a learning Bayesian network algorithm.

In one embodiment of the method according to the present invention, the genotype data comprises allelic data of genes.

This allelic data comprises in one embodiment Single-Nucleotide Polymorphism data.

In an embodiment of the method according to the present invention, the genotype data is extracted which has a maximum probability to correspond to a predetermined set of phenotype features.

A group of genes is selected in one embodiment of the method according to the present invention from all genes of said organism according to a relevance of said group of genes to a predetermined function of said organism.

This function is in one embodiment a cell function of said organism.

Said function is in an alternative embodiment a body function of said organism.

In an embodiment of the method according to the present invention, a list of genes is generated which are related to at least one genetic pathway of said organism.

In an embodiment of the method according to the present invention depending on the locus of the genes on a chromosome, Single-Nucleotide Polymorphisms are extracted which are located on or close to said genes.

In an embodiment of the method according to the present invention, the extracted Single-Nucleotide Polymorphism data is categorized.

In an embodiment of the method according to the present invention, the organisms of the group of organisms are automatically clustered into subgroups of organisms on the basis of the genotype data.

In one embodiment of the method according to the present invention, the clustered organisms are then automatically classified on the basis of the phenotype data.

In one embodiment of the method according to the present invention, the organisms are classified into risk groups for different diseases.

In an alternative embodiment, the organisms are classified into drug response groups for different drugs.

The organisms are either human beings, micro-organisms, animals or plants.

For a better understanding of the nature and advantages of the present invention, it will be described in detail with reference to the enclosed drawings.

### BRIEF DESCRIPTION OF THE DRAWINGS

FIG. 1 shows a block diagram of a computer system for extracting the genotype-phenotype relationship according to one embodiment of the present invention;

FIG. 2 shows a flow chart of an embodiment of the method for extracting a genotype-phenotype relationship according to the present invention;

FIG. 3 shows a table of an example for genotype data and phenotype data for illustrating the functionality of the method according to the present invention;

FIG. 4 shows a first diagram for illustrating the functionality of the method for extracting a genotype-phenotype relationship according to the present invention;

FIG. 5 shows a second diagram for illustrating the functionality of the method for extracting a genotype-phenotype relationship according to the present invention;

FIG. 6 shows a third diagram for illustrating the functionality of the method for extracting a genotype-phenotype relationship according to the present invention.

### DETAILED DESCRIPTION OF PREFERRED EMBODIMENTS

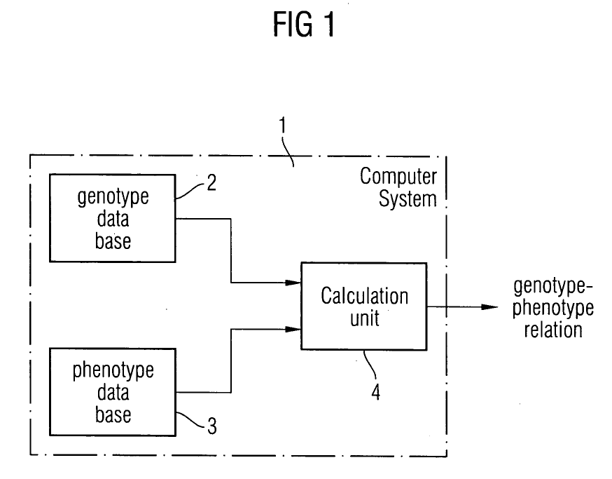

As can be seen from FIG. 1, a computer system 1 according to the present invention for extracting at least one genotype-phenotype relationship comprises at least one genotype database 2 and at least one phenotype database 3. The databases 2, 3 can be public databases or user defined databases. Both databases 2, 3 are connected to a calculation unit 4, such as a computer which outputs the calculated genotype-phenotype relationship to the user. The databases 2, 3 store data of a big number of organisms which are connected to the calculation unit 4 directly or via a network. The phenotype database comprises in one embodiment clinical data of a hospital. The genotype database is in one embodiment a SNP database storing mutational data.

Data entries, such as in a SNP database are in a preferred embodiment based on a tab-delimited txt format and includs:
a unique genetic variation ID
a reference to a public database and public ID
characterization of class of variation for SNPs:
dbSNP ss#
dbSNP rs#
the observed alleles at a particular locus
the 5′ flanking sequence that surrounds the mutation
the 3′ flanking sequence that surrounds the mutation
pointer to genetic map information
the experimental method(s) used to assay the variation
population-specific frequency information
individual-specific genotype information
a pointer to a companion dbSTS or GenBank record
known genes in the region
validation information to describe the quality of the frequency information

Additional data entries in the SNP database are in a preferred embodiment:
the chromosome on which the SNP is located
genomic position on the indicated chromosome
cDNA position
Polymorphism classification, either SNP, in-del (insertion/deletion), MNP (multiple nucleotide polymorphism), or mixed
SNP production method
SNP detection method
SNP validation method

In a preferred embodiment, the databases 2, 3 have the following functionality:
SNP Execution of web-service based retrieval of public SNP data from dbSNP and HapMap
Execution of download of dbSNP XML (NSE) formatted files. Storage on the local hard drive
Execution of download of XML (NSE) files via ftp
Execution of download of bulk data from the HapMap database
Execution of HapMap search, download of HapMap tab delimited txt file and storage on the local hard drive
Conversion of tab delimited HapMap file to XML
Conversion of user-specified private SNP data from a submission form to XML
Upload/storage of SNP data in XML format to the database

The genotype database stores information about identified SNP or haplotype wherein the information is linked to a certain organism or patient and vice versa. To extract a genotype-phenotype relationship for different organisms of a group of organisms the genotype data of each organism of said group is stored in at least one genotype database 2. For each organism, a genotype vector is stored having a vector component for each gene of the group of genes which have to be investigated.

Phenotype data of each organism of said group is stored in at least one second phenotype database 3, wherein for each organism a phenotype vector is stored having a vector component for each phenotype feature of a group of phenotype features of said organism. The calculation unit 4 classifies by a machine learning process organisms with different phenotypes depending on the genotype vectors stored in the first database 2 and on the phenotype vectors stored in the second database 3 to extract the genotype-phenotype relationship which is output to the user.

In one embodiment, the machine learning process is a learning Bayesian network algorithm.

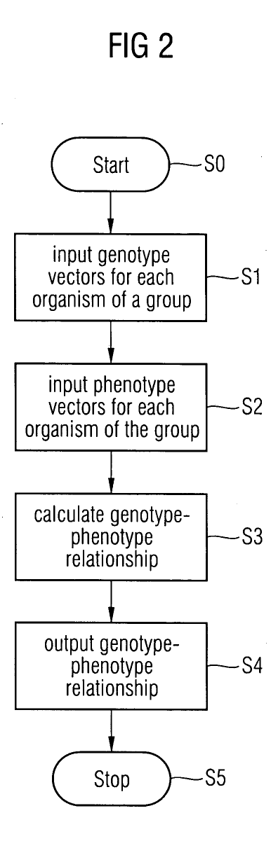

FIG. 2 shows a flow chart of an embodiment of the method according to the present invention. After starting the method in step S0, the genotype vectors of each organism of the investigated group are input in step S1, i. e. the calculation unit 4 reads the genotype vectors from the genotype database 2.

In a step S2, the genotype vectors of each organism of the selected group is input, i. e. the calculation unit 4 reads the phenotype vectors from the phenotype database 3.

In a step S3, the calculation unit 4 calculates a genotype-phenotype relationship in a machine learning process on the basis of the input genotype vectors and the input genotype vectors.

In a step S4, the found genotype-phenotype relationship is output to the user.

The process stops in step S5.

To extract the desired genotype-phenotype relationship, the calculation unit 4 performs a machine learning algorithm. In one embodiment, the machine learning process is a learning Bayesian network algorithm. Each node of the network corresponds to a polymorphism, a gene or a phenotype feature, i. e. to each vector component. The learning Bayesian network algorithm performs a statistical relationship between nodes, graphically represented by edges having corresponding probability values. Methods for learning statistical structures of data are described in “Identifying interventional and pathogenic mechanisms by generative Inverse Modeling of Gene Expression Profiles” by Mathäus Dejon, Martin Stetter et al. in Journal of Computational Biology, Volume 11, Number 6, pp 1135-1148, 2004.

The joint probability density function of the expression levels can be decomposed into the product form

$$P ( {X}_{1} , {X}_{2} , … , {X}_{n} ) = {\prod }_{i = l}^{n} P ( {X}_{i} \vert P {a}_{i} , Θ , G ) , \qquad ( 1 )$$

where the parents Paᵢ of Xᵢ are the set of nodes having a directed edge to gene i.

The procedure of structural learning is stated as follows: When D={d¹,d², . . . ,d^{N}} is a data set of N independent observations, where each data point is an n-dimensional vector with components d¹={d₁^{l}, . . . ,dₙ^{l}} for a given D, a graph structure G and parameters Θ of the Bayes-net B are found that best matches D. To evaluate the quality of a fit of a network with respect to the data, the Bayesian Dirichlet equivalent (BDe) score is used, which is proportional to the posterior probability of G given D:

$$S ( G \vert D ) = \frac{P ( D \vert G ) P ( G )}{P ( D )} , \qquad ( 2 )$$

where P(D|G) is the marginal likelihood, P(G) the prior probability of the structure, and P (D) a normalization constant. Using an uniform prior over possible structures, the learning problem is reduced to finding the structure with the best marginal likelihood according to the data:

P(D|G)=∫P(D|Θ,G)P(Θ|G)dΘ, (3)

where P(Θ|G) denotes the prior density of the model parameters Θ for given G. Given a multinomial model of n variables and other assumptions Equation (3) is solved in closed form:

$$P ( D \vert G ) = {\prod }_{i = 1}^{n} {\prod }_{j = 1}^{qi} \frac{Γ ( {N}_{ij}^{′} )}{Γ ( {N}_{ij}^{′} + {N}_{ij} )} {\prod }_{k = 1}^{{r}_{i}} \frac{Γ ( {N}_{ijk}^{′} + {N}_{ijk} )}{Γ ( {N}_{ijk}^{′} )} , \qquad ( 4 )$$

where rᵢ is the number of values which variable Xᵢ can assume and qᵢ the number of values of Paᵢ; Nᵢⱼₖ denotes the number of cases in data set D in which dᵢⁱ=k and

$$P {a}_{i} ( {d}^{l} ) = j ; {N}_{ij} = {\sum }_{k = 1}^{{r}_{i}} {N}_{ijk} \cdot {N}_{ijk}^{′}$$

express parameters of the Dirichlet prior distributions; and Nᵢⱼ′=ΣₖN′ᵢⱼₖ.

For finding the optimal structure of a Bayesian network, a heuristic search strategy is adopted which efficiently determines a Bayesian network close to the optimum. In one embodiment, simulated annealing (SA) as a local search strategy is applied.

Being a density estimator, the trained Bayesian network B=(G,Θ), Equation (1), is used as a generative probabilistic model to produce a data set D_{g} that mirrors the probability distribution, learned previously from the original data set D. Drawing gene expression profiles without an intervention works as follows (cf. Algorithm 1): First, all variables are ordered such that the parents Paᵢ of each variable Xᵢ are instantiated before Xᵢ itself. Next, variables are selected according to this ordering and instantiated with a value, Xᵢ=X_{i,g}. The value of each variable is selected with a probability P(Xᵢ|Pa_{i,g}), where Pa_{i,g} denotes the selected states for Xᵢ's parents. This procedure is repeated until all variables are instantiated to form a generated global gene expression profile X_{g} and until N gene expression patterns are drawn to form an artificial data set D_{g}.

### Algorithm 1. Sampling (B, N)

### Input:

B-Bayes-net;
N-Number of independent samples.

### Output:

D_{g}-Data set of N independent samples.
   1. Order variable-set X consistent with the condition that parents Paᵢ are sorted before Xᵢ
   2. For s=1, . . . ,N
   3. For i=1, . . . ,n
   4. Let Xᵢ be the highest ordered node not instantiated in sample s
   5. Select state with probability P(Xᵢ=state|Pa_{i,g},E)
   6. Update s-th sample of D_{g}|E
   7. Instantiate Xᵢ=state

To assess the variability of the training data, expression patterns are drawn from all Q graph structures obtained from the bootstrap procedure, until D_{g} is complete.

The approach of interventional modeling estimates the effect of a certain intervention on the behavior of the Bayes-net using a combination of probabilistic inference and data sampling. The aim is to draw gene-expression patterns and to form an artificial data set D_{g|E} under a set of interventions, which are imposed as a set of evidences E. Possible interventions can be, for example, (i) clamping a subset X_{E} of genes to certain values and/or (ii) clamping parts of the graph structure G to certain values yielding a new posterior distribution P′(G)≠P_{Q}(G).

### Algorithm 2. Interventional Sampling (B, E, N)

### Input:

B-Bayes-net;
E-Set of interventions;
N-Number of independent samples.

### Output:

D_{g|E}-Data set of N independent samples given E.
X_{E} Set of observed variables;
X_{q}={X\X_{E}}-Set of query variables.
   1. Order X_{q} consistent with the condition that parents Paᵢ are sorted before Xᵢ
   2. For s=1, . . . ,N
   3. For i=1, . . . ,n
   4. Let Xᵢ be the highest ordered node not instantiated in sample s
   5. Select state with probability P(Xᵢ=state|Pa_{i.g},E)
   6. Update s-th sample of D_{g}
   7. Instantiate Xᵢ=state

Generating data under interventions (cf. Algorithm 2) is done by propagation of evidence through the Bayes-net, that is, by obtaining the posterior distributions of the subset X_{q}=X\X_{E} of free expression levels. The posterior distribution follows

$$P ( {X}_{q} \vert E , ) = {\sum }_{G} P ( {X}_{q} \vert E , G ) {P}^{′} ( G ) , \qquad ( 5 )$$

where P(X_{q}\E,G) denotes the joint probability to measure gene expression levels X_{q} in a network with structure G, given certain genes have been fixed to expression levels by an intervention E. Before instantiation, the free variable set X_{q} is sorted as described in the previous section, such that for each variable XᵢεX_{q} its parents Paᵢ are ordered before the variable itself. In contrast to the sampling procedure without intervention, the distribution over values of Xᵢ depend on its parents Paᵢ and on the set of intervention E.

Thus, the conditional probability has to be calculated performing Bayesian inference

$$P ( {X}_{i} \vert P {a}_{i , g} , E ) = \frac{P ( {X}_{i} , P {a}_{i , g} , E )}{P ( P {a}_{i , g} , E )} , \qquad ( 6 )$$

where the numerator is computed by marginalizing the joint distribution and the denominator is obtained by a subsequent marginalization over Xᵢ:

$$P ( {X}_{i} \vert P {a}_{i , g} , E ) = \frac{{\sum }_{X ∖ {X}_{i} , P {a}_{i} , {X}_{E}} P ( X )}{{\sum }_{X \backslash {X}_{g} , P {a}_{i}} P ( X )} . \qquad ( 7 )$$

In order to efficiently solve Equation (7), bucket elimination is used, i. e. an exact inference algorithm in which variables are summed out one at a time. Each gene XᵢεX_{q} is then instantiated according to Equation (7) until the full vector X=(X_{q},X_{E}) of gene-expression levels is instantiated.

In alternative embodiments, the machine learning process is performed by a support vector machine or by a neural network or a decision tree or fuzzy system.

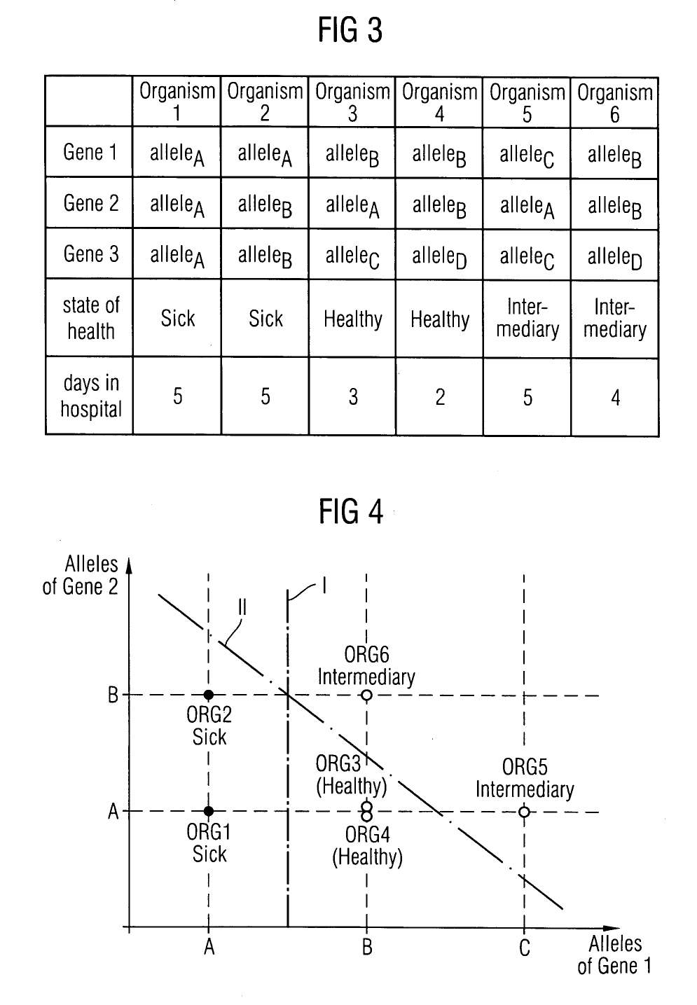

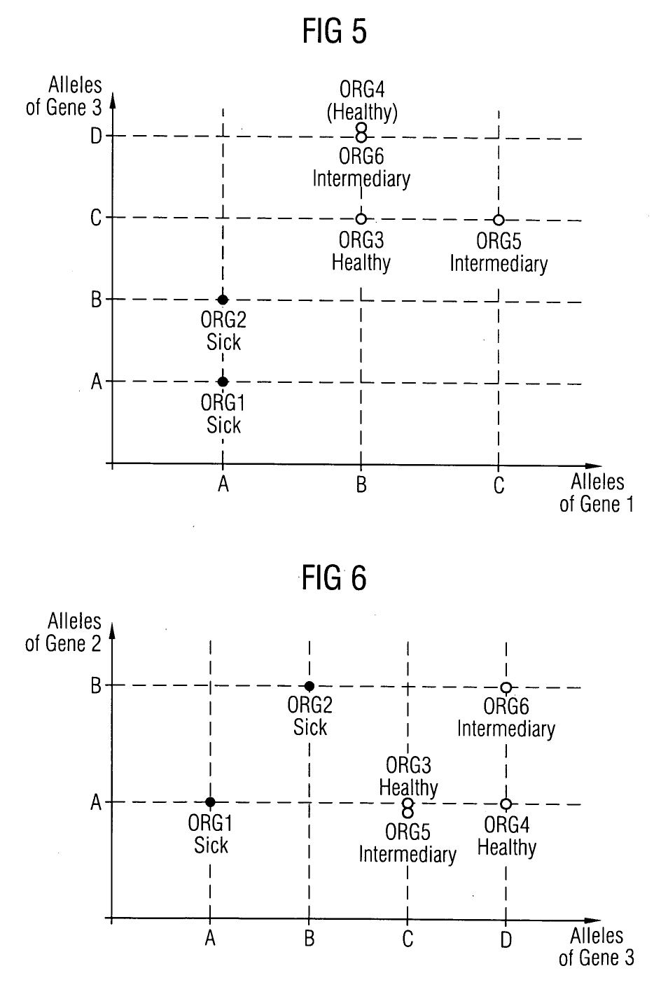

To demonstrate the functionality of the method according to the present invention, it will be described the following to a simple example as shown in FIGS. 3 to 6.

FIG. 3 shows a table stored in a database holding genotype data and phenotype data for six different organisms of a group of organisms.

In the given example, the genotype data is formed by allelic data indicating different alleles of a gene. An allele is anyone of a number of alternative forms of the same gene occupying a given local on a chromosome of said organism. An allele is a variation of a gene. The genes stored in the database can be all genes of the organism. However, in most cases, genes which are investigated for extracting the desired genotype-phenotype relationship are a selected group of genes. Normally, the genes are selected from all genes of said organism according to a relevance of said group of genes to a predetermined function of the organism. This function is either a cell function or a body function of the organism. For instance, all genes which are involved in a biological pathway of said organism are selected to extract the genotype-phenotype relationship. In the example shown in FIG. 3, the genotype data of all organisms for three genes, i. e. gene 1, gene 2, gene 3, are provided for extracting the desired genotype-phenotype relationship.

The phenotype data of said organisms are for instance clinical data, such as shown in FIG. 3. In the given example, the clinical data indicates the state of health of the different organisms 1-6 which might be patients, i. e. human beings treated in a hospital. The genotype data of the example shown in FIG. 3 further indicates the number of days the patient stayed in the hospital.

The phenotype data and the genotype data are provided to the calculation unit 3 from the databases 2, 3 as genotype vectors and phenotype vectors. Each genotype vector comprises at least one vector component for each gene of the selected group of genes. Furthermore, each phenotype vector comprises a vector component for each phenotype feature of the user defined group of phenotype features of the patients.

For the given example, the following genotype and phenotype vectors can be defined as:

$${}_{{genotype}_{ORG 1}} = ( \begin{matrix}A \\ A \\ A\end{matrix} ) \qquad {}_{{phenotype}_{ORG 1}} = ( \begin{matrix}S \\ 5\end{matrix} )$$

$${}_{{genotype}_{ORG 2}} = ( \begin{matrix}A \\ B \\ B\end{matrix} ) \qquad {}_{phenotypeORG 2} = ( \begin{matrix}S \\ 5\end{matrix} )$$

$${}_{{genotype}_{ORG 3}} = ( \begin{matrix}B \\ A \\ C\end{matrix} ) \qquad {}_{{genotype}_{ORG 3}} = ( \begin{matrix}H \\ 3\end{matrix} )$$

$${}_{{genotype}_{ORG 4}} = ( \begin{matrix}B \\ B \\ D\end{matrix} ) \qquad {}_{{phenotype}_{ORG 4}} = ( \begin{matrix}H \\ 2\end{matrix} )$$

$${}_{{genotype}_{ORG 5}} = ( \begin{matrix}C \\ A \\ C\end{matrix} ) \qquad {}_{{phenotype}_{ORG 5}} = ( \begin{matrix}I \\ 5\end{matrix} )$$

$${}_{{genotype}_{ORG 6}} = ( \begin{matrix}B \\ B \\ D\end{matrix} ) \qquad {}_{{phenotype}_{ORG6}} = ( \begin{matrix}I \\ 4\end{matrix} )$$

For example, the genotype vector for organism 1 is (AAA) indicating that the organism has the allele A for each of the genes 1-3. The phenotype vector of organism 1 is (S5) indicating that the organism is sick (S) and had to stay five days in the hospital.

The FIG. 4 shows the first diagram for illustrating the method according to the present invention. As can be seen from FIG. 4, the alleles of gene 1 and of gene 2 are arranged in two dimensions according to the different allelic data. Organisms 1-6 are automatically clustered into subgroups of organisms on the basis of the input genotype data. Alternatively, the organisms are classified on the basis of the phenotype data.

As can be seen from FIG. 4, the first classification distinguishes the sick organisms from the other organisms by a line I.

Another classification line II delimits the healthy organisms 3, 4 from the other organisms.

With classification of the investigated organisms it is possible to extract automatically the genotype-phenotype relationship on the basis of the input genotype data for the collected group of genes for different organisms of the investigated group of organisms.

In the given example, the first genotype-phenotype relationship extractable on the basis of the given data is:

If gene 1=allele A, then the organism is sick.

A second genotype-phenotype relationship which is extractable on the basis on the given data is:

If gene 1 is B and if gene 2 is A, then the organism is healthy.

In a preferred embodiment of the method according to the present invention, the found genotype-phenotype relationship is given with a certain probability.

For instance, in the given example, the probability for the genotype-phenotype relationship:

“If gene 1 is B and gene 2 is A, then the organism is healthy” is 100%.

In one embodiment, the organisms are classified into drug response groups for different drugs. In an alternative embodiment, the organisms are classified into risk groups for different diseases, such as cancer. The investigated organisms for which a genotype-phenotype relationship is extracted according to the present invention are any kind of organism, such as human beings, micro-organisms, animals and plants.

In one embodiment of the method according to the present invention, a list of genes is generated which are related to at least one genetic pathway of the organism. Depending on the locations of the genes, a search function is activated which is looking for SNPs located on or close to those genes. These SNPs are then categorized into coding/non-coding, located in an enhancer/promoter region, an intron/exon location, non-synonymous or synonymous or categorized in missense/nonsense. The result is a list of all factors which potentially cause changes in the genetic network and might contribute to diseases. The user has the option to tailor the list to experiment related requirements. The user can consider the whole list for subsequent tasks or only a subset of it.

For connecting the sequence information with the gene expression and also the phenotype of a certain population, a query function is provided. The query function represents a logical combination of SNP states and the search function looks for organisms which have certain user defined overlap of their respective alleles with the SNP patterns. In case that the search results match, the organism is considered to be a positive individual actually having a certain pattern. On the other hand, in case that an organism's allele pattern does have no or very little overlap, the given SNP pattern firstly is considered to be a negative individual. Thus, the method according of the present invention extracts subsets out of the original patient population which allows a study of changes in the genetic networks and the phenotype subsequently. The search and retrieval function allows a patient stratification and provides an important capacity for all SNP related statistical analysis, e. g. pre-dispositions or diseases like cancer or potential individual side effects of drugs.

In an embodiment of the method according to the present invention the method is performed by a computer program stored on a data carrier.

What is claimed is:

1. A method for extracting at least one genotype-phenotype relationship on the basis of genotype data of a group of genes or polymorphisms for different organisms of a group of organisms;
(a) wherein genotype data of each organism of said group of organisms is input as a genotype vector having at least one vector component for each gene of said group of genes or for each polymorphism;
(b) wherein phenotype data of each organism of said group of organisms is input as a phenotype vector having a vector component for each phenotype feature of a group of phenotype features of said organism;
(c) wherein by a machine learning process organisms with different phenotypes are classified depending on said input genotype vectors and said input phenotype vectors to extract said genotype-phenotype relationship.

2. The method according to claim 1, wherein said machine learning process is a learning Bayesian network algorithm.

3. The method according to claim 1, wherein said genotype data comprises allelic data of said gene.

4. The method according to claim 3, wherein said allelic data comprises Single-Nucleotide Polymorphism-(SNP) data.

5. The method according to claim 1, wherein the genotype data is input from a first database.

6. The method according to claim 1, wherein the allelic data indicates alternative forms of said gene occupying a predetermined locus on a chromosome of said gene.

7. The method according to claim 1, wherein genotype data is extracted which has a maximum probability to correspond to a predetermined set of phenotype features.

8. The method according to claim 1, wherein said phenotype data is input from a second database.

9. The method according to claim 1, wherein said group of genes is selected from all genes of said organism according to a relevance of said group of genes to a predetermined function of said organism.

10. The method according to claim 7, said function is a cell function of said organism.

11. The method according to claim 7, wherein said function is a body function of said organism.

12. The method according to claim 1, wherein a list of genes is generated which are related to at least one genetical pathway of said organism.

13. The method according to claim 1, wherein depending on the locus of said genes on a chromosome Single-Nucleotide Polymorphisms are extracted which are located on or close to said genes.

14. The method according to claim 1, wherein the extracted SNP are categorized.

15. The method according to claim 1, wherein the organisms of said group of organisms are automatically clustered into subgroups of organisms on the basis of said genotype data.

16. The method according to claim 15, wherein the clustered organisms are automatically classified on the basis of said phenotype data.

17. The method according to claim 1, wherein the organisms are automatically classified on the basis of said phenotype data.

18. The method according to claim 17, wherein the organisms are classified into risk groups for different diseases.

19. The method according to claim 17, wherein the organisms are classified into drug response groups for different drugs.

20. The method according to claim 1, wherein the organisms are formed by human beings.

21. The method according to claim 1, wherein the organisms are formed by microorganisms.

22. The method according to claim 1, wherein said organisms are formed by animals.

23. The method according to claim 1, wherein said organisms are formed by plants.

24. The method according to claim 1, wherein the machine learning process is a supervised learning process.

25. The method according to claim 1, wherein the machine learning process is an unsupervised machine learning process.

26. A computer Program for extracting at least one genotype phenotype relationship on the basis of genotype data of a group of genes or polymorphisms for different organisms of a group of organisms comprising the following steps:
(a) reading genotype data of each organism of said group of organism as a genotype vector having at least one vector component for each gene of said group of genes or for each polyphormism;
(b) reading phenotype data of each organism of said group of organism as a phenotype vector having a vector component for each phenotype feature of a group of phenotype features of said organism;
(c) classifying by means of a machine learning algorithm organisms with different phenotypes depending on the read genotype vectors and the read input phenotype vectors to extract said genotype-phenotype relationship.

27. A data carrier for extracting at least one genotype-phenotype relationship on the basis of genotype data of a group of genes or polymorphisms for different organisms of a group of organism, said computer program comprising the following steps:
(a) reading genotype data of each organism of said group of organism as a genotype vector having at least one vector component for each gene of said group of genes or for each polymorphism;
(b) reading phenotype data of each organism of said group of organism as a phenotype vector having a component for each phenotype feature of a group of phenotype features of said organism;
(c) classifying by means of a machine learning algorithm organisms with different phenotypes depending on the read genotype vectors and the read input phenotype vectors to extract said genotype-phenotype relationship.

28. Computer system for extracting at least one genotype-phenotype relationship on the basis of genotype data of a group of genes or polymorphisms for different organisms of a group of organisms, said computer system comprising:
(a) a first database for storing genotype data of each organism of said group of organism, wherein for each organism a genotype vector is stored having at least one vector component for each gene of the group of genes or for each polymorphism;
(b) a second database for storing phenotype data of each organism of said group of organism, wherein for each organism a phenotype vector is stored having a vector component for each phenotype feature of a group of phenotype features of said organism;
(c) a calculation unit which classifies by a machine learning process organisms with different phenotypes depending on the genotype vectors stored in said first database and on the phenotype vectors stored in said second database to extract the genotype-phenotype relationship.
